(19) United States
(12) Patent Application Publication
(10) Pub. No.: US 2009/0291108 A1
(43) Pub. Date: Nov. 26, 2009

(54) Fabric with health care component

(76) Inventors: Tsai-Chin Chen, Taichung (TW); Wei-Ting Lu, Taichung (TW)

Correspondence Address: SAM CHEN, 7F-1, 293, ROOSEVELT ROAD, SEC 3, TAIPEI, TW

(21) Appl. No.: 12/126,921

(22) Filed: May 25, 2008

(51) Int. Cl. A61K 9/70; A61P 25/00

(52) U.S. Cl. 424/402

(57) ABSTRACT

A fabric includes therapeutic members formed thereon, the therapeutic member comprising powder including Ge, GeO₃, Al₂O₃, and P for generating and discharging negative ions; MgO, TiO₂, and ZrO for emitting FIR radiant heat; Fe for discharging static electricity; and ceramic for generating FIR energy and blocking harmful UV rays; an adhesive mixed with the powder for fixedly secure the therapeutic member onto the fabric; and a dilution agent mixed with the powder and the adhesive for preventing the therapeutic members from being glued together when the fabric is folded. The fabric is adapted to make, for example, an article of clothing, a head band, or a wrist band.

### BACKGROUND OF THE INVENTION

1. Field of Invention

The invention relates to health care materials and more particularly to a fabric having therapeutic members formed thereon such that the therapeutic members on an article of clothing or wearing made from the fabric may create FIR waves and negative ions for relieving pain and obtaining other health care benefits.

2. Description of Related Art

Infrared (IR) energy utilized for heating is well known. Far infrared (FIR) energy as a branch of IR energy is beneficial for a wide range of ailments is also known. It is found that FIR waves having the wavelength in the range of 4 μm to 14 μm are particularly beneficial for health.

There have been numerous suggestions in prior patents for infrared based health care devices. For example, U.S. Pat. No. 6,510,346 discloses a therapeutic infrared energy wrist cover.

It is also known that negative ions containing a small percentage of ozone are capable of destroying disease-producing bacteria. Hence, devices for generating negative ions have been commercially available in recent years and are becoming increasingly popular.

Unfortunately, the commercially available negative ion generators are high in price and its functions are also limited. Moreover, its manufacturing processes are complicated. Thus, the need for improvement still exists.

### SUMMARY OF THE INVENTION

It is therefore one object of the invention to provide a fabric having therapeutic members formed thereon such that the therapeutic members on an article of clothing or wearing made from the fabric may create FIR waves and negative ions for relieving pain and obtaining other health care benefits.

The above and other objects, features and advantages of the invention will become apparent from the following detailed description taken with the accompanying drawings.

### BRIEF DESCRIPTION OF THE DRAWINGS

FIG. 1 is a perspective view of a portion of fabric according to the invention;

FIG. 2 is a longitudinal sectional view of the fabric of FIG. 1;

FIG. 3 is a front view of a dress made of the fabric according to a first preferred embodiment of the invention;

FIG. 4 is a front view of a wrist band made of the fabric according to a second preferred embodiment of the invention;

FIG. 5 is a front view of a head band made of the fabric according to a third preferred embodiment of the invention; and

FIG. 6 is a fragmentary view of FIG. 2 schematically showing FIR waves and negative ions being created by the therapeutic member.

### DETAILED DESCRIPTION OF THE INVENTION

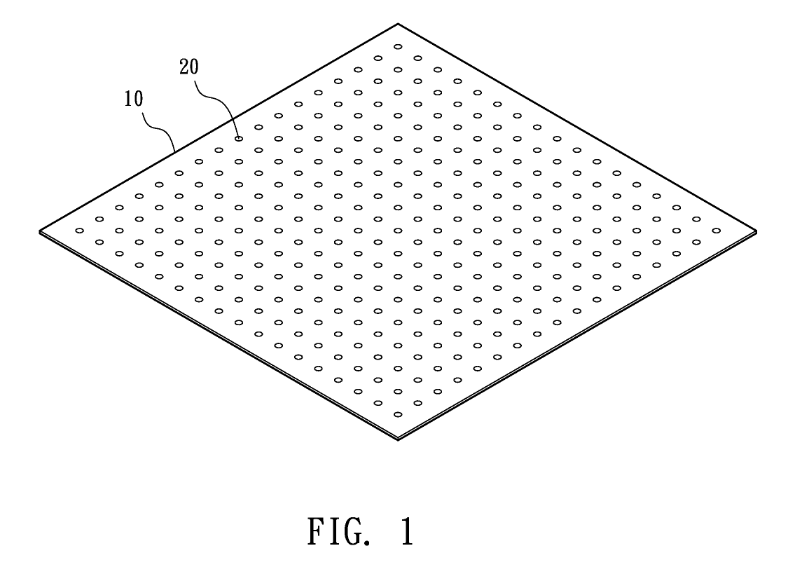

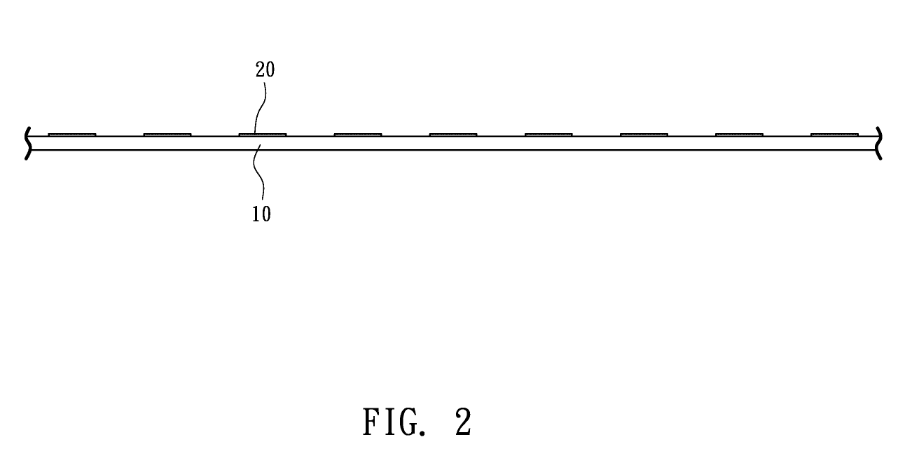

Referring to FIGS. 1 and 2, a fabric in accordance with the invention is generally designated 10. The fabric 10 is made from fibers, cotton, wool, silk, threads, artificial leather, paper, plastics, or nylon. The fabric 10 has a plurality of therapeutic members 20 formed on one surface of the fabric 10 in rows and columns.

The therapeutic member 20 is comprised of mineral powder, adhesive, and dilution agent mixed together. The powder comprises germanium (Ge), GeO₃, Al₂O₃, and phosphorus (P) for generating and discharging negative ions; magnesium oxide (MgO), titanium dioxide (TiO₂), and zirconium oxide (ZrO) for emitting FIR radiant heat; iron (Fe) for discharging static electricity; and ceramic for generating FIR energy and blocking harmful UV (ultraviolet) rays.

The adhesive is adapted to fixedly secure the therapeutic member 20 onto the fabric 10. The dilution agent is adapted to prevent the therapeutic members 20 from being glued together when an article made from the fabric 10 is folded. Moreover, the dilution agent is adapted to enhance the fastening of the therapeutic member 20 onto the fabric 10.

The therapeutic members 20 are formed on the surface of the fabric 10 by pressing or the like. Further, the therapeutic members 20 may be arranged as a logo, mark, or the like for advertisement.

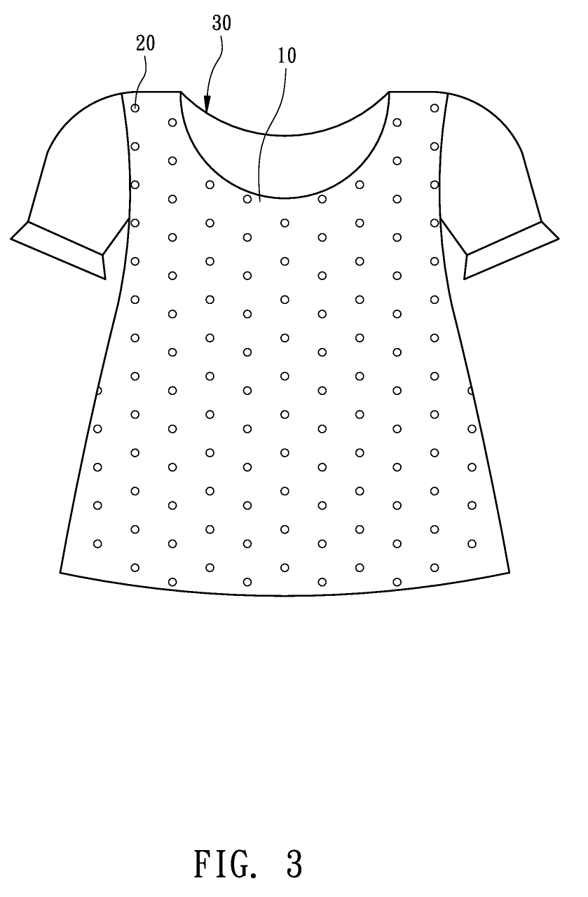

Referring to FIG. 3, a dress 30 made of the fabric 10 according to a first preferred embodiment of the invention is shown.

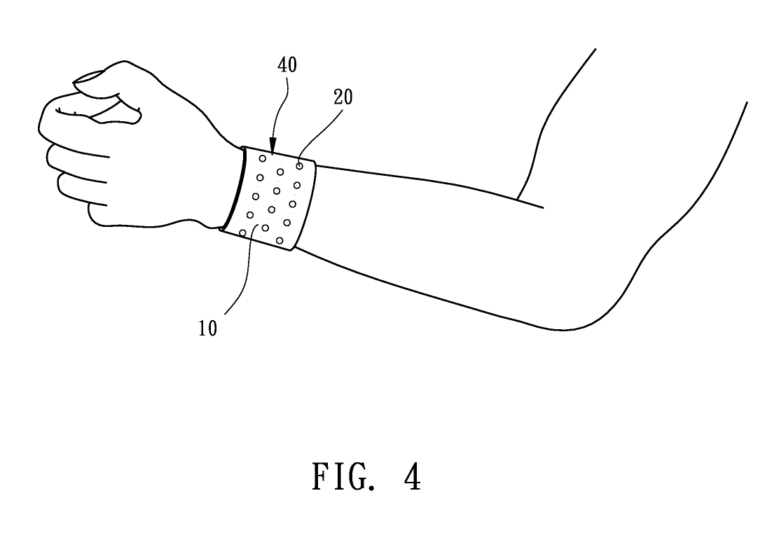

Referring to FIG. 4, a wrist band 40 made of the fabric 10 according to a second preferred embodiment of the invention is shown.

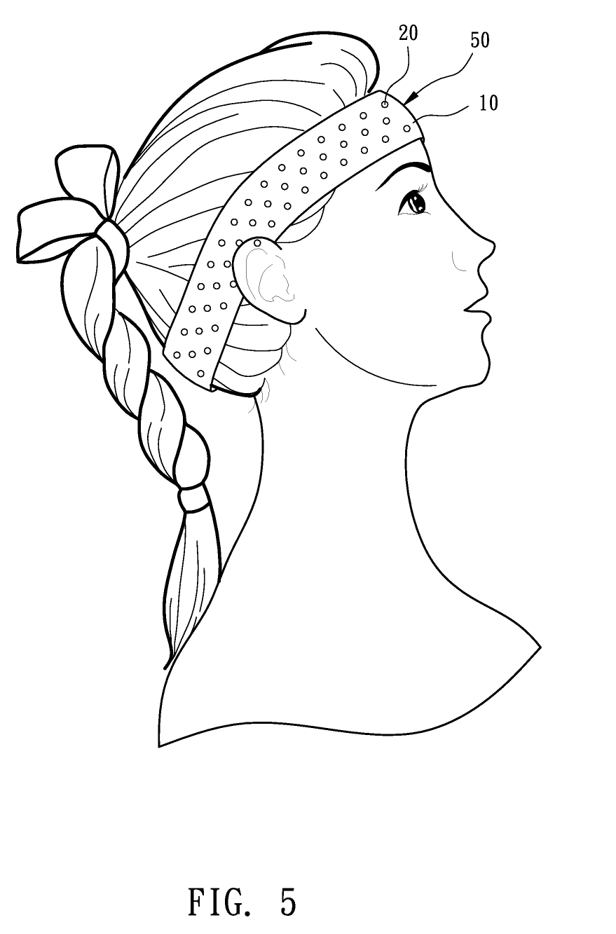

Referring to FIG. 5, a head band 50 made of the fabric 10 according to a third preferred embodiment of the invention is shown.

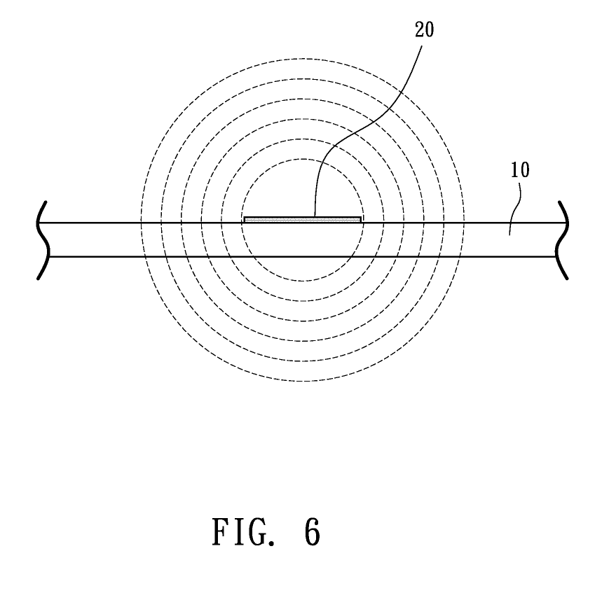

Referring to FIG. 6, it schematically shows FIR waves and negative ions being created by the therapeutic member 20.

It is envisaged by the invention that FIR radiant heat emitted by the fabric 10 is beneficial for health. Also, the negative ions discharged by the fabric 10 are adapted to relieve pain. In short, many health benefits can be obtained if an individual wears an article made from the fabric 10.

While the invention herein disclosed has been described by means of specific embodiments, numerous modifications and variations could be made thereto by those skilled in the art without departing from the scope and spirit of the invention set forth in the claims.

What is claimed is:

1. A fabric including a plurality of therapeutic members formed thereon, the therapeutic member comprising:
powder including germanium (Ge), GeO₃, Al₂O₃, and phosphorus (P) for generating and discharging negative ions; magnesium oxide (MgO), titanium dioxide (TiO₂), and zirconium oxide (ZrO) for emitting FIR (far infrared) radiant heat; iron (Fe) for discharging static electricity; and ceramic for generating FIR energy and blocking harmful UV (ultraviolet) rays;
an adhesive mixed with the powder for fixedly secure the therapeutic member onto the fabric; and
a dilution agent mixed with the powder and the adhesive for preventing the therapeutic members from being glued together when the fabric is folded.

2. The fabric of claim 1, wherein the therapeutic members are arranged as a predetermined shape.

3. The fabric of claim 1, wherein the fabric is adapted to make an article of clothing.

4. The fabric of claim 1, wherein the fabric is adapted to make a head band.

5. The fabric of claim 1, wherein the fabric is adapted to make a wrist band.
